(19) United States
(12) Patent Application Publication
(10) Pub. No.: US 2011/0086385 A1
(43) Pub. Date: Apr. 14, 2011

(54) PROCESS FOR THE PRODUCTION OF BIOGAS

(75) Inventors: Stefan Kromus, Wien (AT); Wilhelmus Antonius Henricus Smeets, Helden (NL); Markus Grasmug, Feldbach (AT)

(73) Assignee: ENBASYS GMBH, Grambach (AT)

(21) Appl. No.: 12/934,502

(22) PCT Filed: Mar. 26, 2009
(86) PCT No.: PCT/AT2009/000121

§ 371 Date: Dec. 23, 2010

(30) Foreign Application Priority Data
Mar. 26, 2008 (AT) A 470/2008

(51) Int. Cl. C12P 1/00; C12M 1/02

(52) U.S. Cl. 435/41; 435/303.3

(57) ABSTRACT

A container (1) for the fermentative production of biogas from organic substrates, which container comprises an axial stirrer (9), one or several inlet devices (2) for filling the container (1), one or several outlet devices (3, 4) for emptying the container (1) and withdrawing a fermentation residue, an external conduit (5) for supplying a fermentation mixture into a closed circular pipeline (7) with several outlets (8) for spraying on the surface (14) of the fermentation mixture, with the sprayed fermentation mixture optionally coming from the lower half of the container (1), a device (11) for withdrawing the biogas produced and an apparatus (10) for controlling the temperature of the fermentation mixture; a process for the fermentative production of biogas from an organic substrate, a process for suppressing foam formation during the fermentative production of biogas, and a process for the improved conversion of oils and fats in organic substrates during the fermentative production of biogas, which can be carried out in a container (1).

The present invention relates to a process and a fermenter for the production of biogas.

Biogas can be obtained by anaerobic fermentation of organic substrates which may come from agriculture, communes and industry. The organic portion which is converted into biogas (such as methane and carbon dioxide) is referred to as degradable CSB (chemical oxygen demand) in anaerobic technology.

A wide variety of organic materials can be treated in an anaerobic reactor. In doing so, different chemical and physical properties arise during the fermentation process due to the composition of the material used. On the one hand, the formation of a gravitational layer may result from heavy solids in the substrate used and, on the other hand, suspended matter as well as oil-containing substances may lead to an accumulation of those substances at the surface. Because of those properties, it is often difficult for the bacterial strains responsible for anaerobic degradation to come into contact with the organic material.

In addition, high organic volume loads often lead to foam formations in the fermenter, whereby the organic volume load can be limited significantly.

Three temperature optima for microorganisms are defined in the anaerobic fermentation: psychrophilic (4-15° C.), mesophilic (20-40° C.) and thermophilic (45-70° C.). The temperature optima differ substantially from the relative growth rates of microorganisms responsible for anaerobic fermentation.

In anaerobic technology, the mesophilic mode of operation generally occurs much more often than the thermophilic one. The reasons are lower energy costs and a higher stability of the process. In numerous studies regarding the thermophilic mode of operation, a higher biochemical reaction velocity, a higher growth rate of microorganisms and a shorter hydraulic retention time were determined. In contrast, however, a higher sensitivity against inhibiting agents such as organic acids, ammonia and hydrogen sulfide exists at higher temperatures, and furthermore, a larger amount of energy is necessary for maintaining the higher temperature.

For substrates having a low CSB concentration (<25 g O₂/l fresh substance), reactor systems such as, e.g., UASB (Upflow Anaerobic Sludge Blanket), EGSB (Expanded Granular Sludge Blanket), IC (Internal Circulation) have been developed which, however, are unsuitable for substrate streams highly concentrated with CSB and having a high particle content and a high oil- and fat-containing portion.

For materials exhibiting a high particle content, a high CSB concentration and a high dry substance content, a “completely stirred tank reactor” (CSTR) or a plug-flow-tank reactor (PFTR dry fermentation systems) may also be used, which must be operated with lower volume loads as compared to those of the above-mentioned fermenter systems in order to be able to ensure an optimum anaerobic degradation for the complex composite substrates. However, due to the low organic volume load possible and the high concentration of substrates, the process procedure is biotechnologically and mechanically limited in size in those systems.

In EP 1 065 268, fermentation tanks with stirrers are described.

In many anaerobic reactors, partly unmixed zones, dead flow spaces in the fermenter, short-circuit flows and floating layers occur. The result is that existing fermenter volumes are often utilized only insufficiently and, respectively, that unfermented substrate leaves the fermenter almost without having been degraded. Furthermore, floating and sediment layers can often be destroyed only with a very large effort.

Reactor systems are also known in which gas or also a liquid is withdrawn from various sites of the fermenter and transferred to other parts of the reactor, e.g., to the head part of the reactor, for better intermixing. However, components (e.g., proteins, fats) may cause massive foam formations particularly at higher volume loads (>6 kg CSB/m³*d) so that also those systems are unable to ensure control of the undesired foam formation.

According to GB 521,036 or EP 0 057 152, it is envisaged that a fermentation liquid is sprayed onto a trickle bed or onto a fermentation liquid above the trickle bed and subsequently is guided across the trickle bed.

According to DE 103 18 298, for example, a fermentation liquid is either pumped from the outside directly into the fermentation liquid in the fermenter or is sprayed from the side onto the surface, and in CN 1 600 749 it is described that a fermentation liquid is to be sprayed circularly into the fermentation tank.

In order to prevent foam formations on the surface, also fermenters having a small surface, for example, egg-shaped fermenters, are used which are employed especially in the anaerobic treatment of sewage sludge coming from the anaerobic wastewater treatment. In the agricultural anaerobic technology, fermenter systems covered with a foil are used in large numbers. It is very difficult to optimally position stirring units because of the large diameters. Furthermore, the fermenter must be emptied for a possible maintenance or repair of the mechanical mixing device and, as a result, the process cannot be operated further so that such systems cannot be used in industrial applications in which residual materials accumulate continuously.

A process for the production of biogas has now surprisingly been found wherein organic substrates rich in solids can be converted continuously at a high concentration and with a high organic volume load, which can be applied with small and large operating volumes, wherein foam formation can be suppressed and which can be applied particularly successfully for organic substrates rich in oil or fat.

In one aspect, the present invention provides
a process for the fermentative production of biogas from an organic substrate
a process for suppressing foam formation during the fermentative production of biogas, or
a process for the improved conversion of oils and fats in organic substrates during the fermentative production of biogas,
   which is characterized in that a fermentation mixture comprising water, an organic substrate and microorganisms is stirred for example continuously or discontinuously in a container with a stirrer axially mounted in the container and that fermentation mixture is conveyed e.g. from the lower half such as the lower third of the container via an external conduit into a closed circular pipeline having several spray nozzles and is sprayed in the container across the surface of the fermentation mixture for example continuously or discontinuously.

The fermentation mixture which is sprayed across the surface preferably comes from the container in which fermentation is carried out, but may also be added from a different fermenter. Preferably, the fermentation mixture comes from the lower half of a fermenter, particularly preferably from the lower third, e.g., from the container in which fermentation is carried out.

### DESCRIPTION OF THE DRAWING

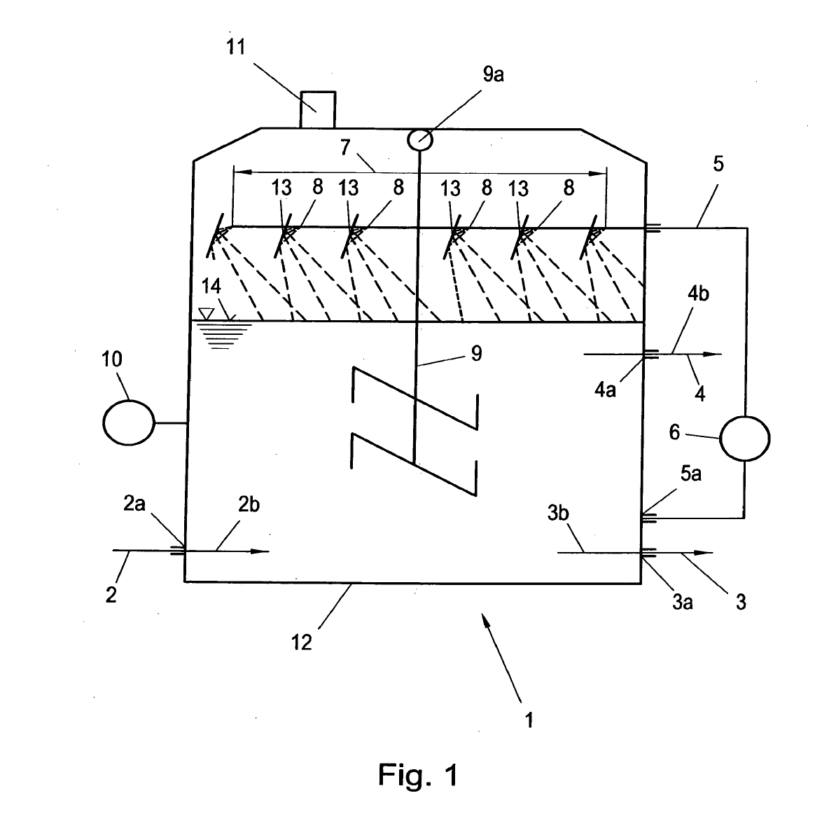

In FIG. 1, a fermenter (1) is schematically shown which comprises an inlet device (2) with an inlet (2a) and an inlet pipe (2b), outlet devices (3, 4) with outlets (3a, 4a) and outlet pipes (3b, 4b), an externally routed pipeline (5), a pump (6), a closed circular pipeline (7) with outlets (8), an axial stirrer (9), a device (10) for controlling the temperature of the fermentation mixture and a device (11) for withdrawing gas.

In another aspect, the present invention provides a container (1) for the fermentative production of biogas from organic substrates, which container comprises an axial stirrer (9), e.g., comprising a driving device (9a), e.g., a motor, one or several inlet devices (2) for filling the container (1) which is/are preferably mounted just above the bottom (12) of the container (1), one or several outlet devices (3, 4) for emptying the container (1) and withdrawing a fermentation residue, for example, an outlet device (3) mounted just above the bottom (12) of the container (1) and a further outlet device (4) mounted in the upper third of the container (1), an external conduit (5), with the inlet (5a) into the external conduit (5) preferably being located in the lower half of the container (1), for supplying a fermentation mixture into a closed circular pipeline (7) with several outlets (8) which are provided, e.g., with spray nozzles and optionally baffle devices (13) for spraying on the surface (14) of the fermentation mixture, a device (11) for withdrawing the biogas produced and an apparatus (10) for controlling the temperature of the fermentation mixture.

In a process according to the present invention, the nature of the organic substrate is of no importance. For example, the organic substrate may optionally comprise pressed organic waste coming, e.g., from waste collection, residual materials from the food processing industry and/or other industrial organic residual materials.

According to the present invention, the degradation of the organic substrate occurs in a fermentative manner, i.e., in the presence of microorganisms, for example, bacteria which are able to break down organic material into biogas such as methane or CO₂. Such bacteria are preferably mesophilic or thermophilic bacteria or mixtures thereof. The process according to the present invention is preferably an anaerobic process.

A container in a process according to the present invention is a fermenter (reactor), preferably a container (1).

A closed circular pipeline (7) comprises a conduit which is mounted above the surface of the fermentation mixture located in the container in such a way that, if possible, the entire surface (14) of the fermentation mixture in the container (1) can be sprayed with more fermentation mixture by means of the spray nozzles at the outlets (8). The closed circular pipeline (7) preferably runs essentially parallel to the surface (14) of the fermentation mixture. The shape of the closed circular pipeline (7) is not crucial, however, the closed circular pipeline (7) should have a shape which does not hamper the throughput of the fermentation mixture, for example, a rounded shape, e.g., circular or oval, or an angular shape, e.g., with 6 or more corners. The outlets (8) are installed on the closed circular pipeline (7) at suitable distances, e.g., at regular distances. Spray nozzles are attached to the outlets (8). “Spray nozzles”, as used herein, include conduits with constrictions at the exit, i.e., nozzles, but also simple conduits without constrictions at the exit through which the fermentation mixture is squeezed under pressure, e.g., by means of the pump (6). “Several spray nozzles” comprise at least 2 spray nozzles, preferably more than 2 spray nozzles, particularly preferably so many spray nozzles that, if possible, the entire surface (14) of the fermentation mixture can be sprayed uniformly. It has become apparent, for example, that excellent results can be achieved with a fermenter having a capacity of approx. 3000 m³ and comprising 6 spray nozzles installed on a hexagonal closed circular pipeline.

Preferably, the jet is guided from a spray nozzle at the outlet (8) to a baffle device (13), e.g., a baffle plate or baffle disk, for example a baffle plate or baffle disk as used in the agricultural discharge of liquid manure, from which the fermentation mixture is sprayed across the surface (14) of the fermentation mixture. By means of the baffle device (13), a particularly good distribution of the sprayed fermentation mixture across the entire surface (14) of the fermentation mixture in the container (1) is achieved. Preferably, fermentation mixture is sprayed onto the surface (14) of the fermentation mixture in the direction of rotation of the stirring device (9). Preferably, the spray nozzles at the outlets (8) and/or the baffle devices (13) are adjusted such that the fermentation mixture sprayed hits the surface (14) of the fermentation mixture in an oblique manner. The spray nozzles at the outlets (8) can be attached to the closed circular pipeline (7) so as to be adjustable, e.g., adjustable in all directions, or in a rigid manner. In one embodiment of the present invention, the spray nozzles at the outlets (8) are attached rigidly, in another embodiment, they are attached so as to be adjustable.

The spraying of the fermentation mixture onto the surface (14) of the fermentation mixture in the container (1) is effected continuously or discontinuously, e.g., discontinuously as soon as foam formation occurs, or continuously, e.g., in cases in which a strong and continuous foam formation occurs and/or in cases in which the organic substrate includes oil- or fat-containing substances floating on the surface (14) of the fermentation mixture in the container (1). In the latter case, a better and quicker conversion of the substrate can be achieved by spraying, since the fermentation mixture sprayed comes into continuous contact with the oil- or fat-containing substances on the surface (14) and, as a result, the degradation thereof can be facilitated and accelerated.

A container (1) includes an apparatus (10) by means of which the temperature of the fermentation mixture can be controlled. Fermentation is preferably carried out in a temperature range lying between the mesophilic and thermophilic fermentation zones, e.g., in a temperature range from 30° C. to 60° C. such as from 40° C. to 50° C.

In a particularly preferred embodiment according to the present invention, a process for the production of biogas is performed as follows, wherein reference is made to FIG. 1:

The supply of the aqueous organic substrate occurs from below through a distribution system (2) lying close to the bottom in order to introduce the substrate into the container (1) largely uniformly across the container cross-section. If required, fermentation mixture is introduced from the lower third of the container (1) into a closed circular pipeline (7) installed above the surface (14) of the fermentation mixture via an externally routed pipeline (5) using a pump (6) and is sprayed across the surface (14) of the fermentation mixture in the container (1) through the spray nozzles at the outlets, which preferably constitute simple conduits without constrictions at the exit, preferably via a baffle device (13). Spraying occurs in the direction of rotation of the axial stirrer (9). The spray nozzles at the outlets (8) and/or the baffle devices (13) are adjusted such that the fermentation mixture sprayed hits the surface (14) of the fermentation mixture in the container (1) in an oblique manner in order to cover, if possible, the entire liquid surface in the reactor. If required, the fermentation mixture in the container (1) is additionally mixed by means of the axial stirrer (9), for example, in case of a strong tendency toward foam formation or a high oil or fat content of the organic substrate. By stirring with the axial stirrer (9), gas bubbles which may adhere to the biomass (partly degraded organic substrate) can be separated from the bacteria more easily and thereby be transported to the liquid surface more easily.

Depending on the substrate (sand, dry substance), the bigger part of the digested sludge (fermentation residue) is withdrawn through outlet devices (3) and (4) installed in the upper third as well as in the lower region of the container (1). Sludge (active sludge, fermentation mixture and microorganisms, in which the fermentation proceeds actively) is normally present at a high concentration in the lower third of the container (1). The substrate degradation in the upper part of the container (1) is enhanced in particular by the fact that concentrated sludge which is introduced into the fermentation mixture in the container (1) through the externally routed pipeline (6) via the closed circular pipeline (7) brings about an increased concentration of active sludge during the settling process and hence a faster substrate degradation in the upper part of the container (1).

By spraying the sludge onto the surface (14) of the fermentation mixture in the container (1), a mechanical destruction of foam which might possibly develop is caused in addition, the effectiveness of which is enhanced by the spray nozzles at the outlets (8), which spray nozzles are preferably installed in an inclined manner toward the stirring direction, optionally in connection with the baffle devices (13) by means of which a particularly good distribution of the sprayed fermentation mixture across the entire surface (14) of the fermentation mixture in the container (1) is effected. A further gain in effectiveness results from the fact that the sprayed sludge has a low pH value which is adjusted by hydrolytic degradation processes in the lower third of the container (1) and from the fact that the low pH value promotes the destruction of foam and the degradation of floatables with an active biomass.

A further parameter in said process is the process temperature which is adjusted by means of the device for controlling the temperature of the fermentation mixture (10), particularly preferably to 40° C.-50° C.

In a container (1) or in a process provided by the present invention, optimum properties (growth rate, degradation of carbohydrates, protein and fat) of mesophilic and thermophilic bacteria are used. Thereby and by a combination with the mechanical devices, the reactor system can be operated at organic volume loads of up to 15 [kg CSB/m³*d].

A container (1) is preferably a container according to FIG. 1.

In a process for suppressing foam formation or in a process for the improved conversion of oils and fats into organic substrates during the fermentative production of biogas according to the present invention, a process which, according to the present invention, is provided for the fermentative production of biogas is preferably used, wherein preferably a container (1) is used.

An advantage of the process for the production of biogas according to the present invention is that it can be employed industrially. A further advantage is that the process can be used for small and large fermentation mixture volumes, e.g., for volumes ranging from 1 m³ to 7000 m³. A further advantage is that the formation of foam can be reduced or prevented, respectively. A further advantage is that the process can be operated at a high nitrogen concentration. It has turned out, for example, that a process according to the present invention for the production of biogas can be operated without any problems at a total nitrogen concentration of up to 9 g TKN (Total Kjeldahl Nitrogen/1 fresh substance) in the organic substrate.

### EXAMPLE

A daily amount of 150 m³ of an organic substrate, consisting of a mixture of squeezed organic waste coming from waste collection, residual materials from the food processing industry and industrial organic residual materials and water, is introduced continuously into a 3000 m³ fermenter having an operating volume of 2850 m³, which is designed according to FIG. 1 and contains bacteria for the anaerobic degradation of an organic substrate in an aqueous fermentation solution. The substrate has a dry substance content of 17% and a CSB concentration of 260 g O₂/kg, resulting in an organic volume load of 14 [kg CSB/m³*d].

The organic substrate is introduced approximately one metre above the fermenter bottom through a distribution system located at the bottom. The biomass concentration in the lower part of the fermenter is higher (sludge bed), whereby the freshly supplied substrate meets a high concentration of active biomass.

A certain amount of said sludge having a higher dry substance content is continuously withdrawn from the lower third of the reactor (V=90 m³), is conveyed to the roof of the fermenter via an external conduit and, using spray nozzles, is sprayed onto the fermentation mixture via a closed circular pipeline in the upper part (in the gas zone) of the fermenter in the direction of rotation of the axial stirrer. Foam (protein-fat compounds) which forms during fermentation is thereby killed and floating substances (e.g., fats and oils, fibrous materials) are contacted with active biomass from the lower part of the reactor. An axial stirrer is used for mechanical stirring. The rotational speed of the stirrer is between 0 and 60 U/min. This stirrer, which is operated discontinuously, serves for an improved release of microbially formed gases (methane, carbon dioxide) in the lower sludge layers and for a dry substance concentration in the entire reactor system which is handled in a controlled manner.

A device (9) for withdrawing gas is located at the highest point of the fermenter.

The withdrawal of the fermentation residue (fermenter content) is effected in the upper third by means of the outlet and the outlet pipe (3) and in the lower third by means of the outlet and the outlet pipe (2) of the fermenter.

The biogas productivity amounts to 5.8 m³ biogas/m³ fermenter volume*d, and the methane content of the biogas ranges from 60% to 65%. The process is operated at a temperature of 40-50° C.

The process is in continuous operation with fermenter systems of 3000 m³ each and yields excellent results.

1. A process for the fermentative production of biogas from an organic substrate, characterized in that a fermentation mixture comprising water, an organic substrate and microorganisms is stirred in a container with a stirrer axially mounted in the container and that fermentation mixture is conveyed via an external conduit into a closed circular pipeline having several spray nozzles and is sprayed across the surface of the fermentation mixture in the container.

2. A process according to claim 1, characterized in that the fermentation mixture which is sprayed comes from the lower third of the container.

3. A process according to claim 1, characterized in that fermentation mixture is sprayed onto the surface of the fermentation mixture in the container in the direction of rotation of the stirrer.

4. A process according to claim 1, characterized in that the fermentation mixture which is sprayed on is sprayed onto the surface of the fermentation mixture via a baffle device.

5. A process according to claim 1, characterized in that the spray nozzles and/or a baffle device is/are adjusted such that the fermentation mixture sprayed hits the surface of the fermentation mixture in the container in an oblique manner.

6. A process according to claim 1, characterized in that fermentation is carried out in a temperature range lying between mesophilic and thermophilic fermentation zones.

7. A process according to claim 6, characterized in that fermentation is carried out in a temperature range from 30° C. to 60° C.

8. A process according to claim 6, characterized in that fermentation is carried out in a temperature range from 40° C. to 50° C.

9. A container for the fermentative production of biogas from organic substrates, comprising an axial stirrer, one or several inlet devices for filling the container, one or several outlet devices for emptying the container and withdrawing a fermentation residue, an external conduit for supplying a fermentation mixture into a closed circular pipeline with several outlets for spraying on the surface of the fermentation mixture, a device for withdrawing the biogas produced and an apparatus for controlling the temperature of the fermentation mixture.

10. A container according to claim 9, characterized in that the outlets are provided with spray nozzles and optionally baffle devices.

11. A container according to claim 10, characterized in that the spray nozzles at the outlets or optionally the baffle device is/are adjusted such that the fermentation mixture sprayed hits the surface of the fermentation mixture in an oblique manner.

12. A container according to claim 10, characterized in that the fermentation mixture sprayed hits the surface of the fermentation mixture in the direction of rotation of the axial stirrer.

13. A container according to claim 10, characterized in that the inlet into the external conduit is located in the lower half of the container.

14. A process for suppressing foam formation or a process for the improved conversion of oils and fats into organic substrates during the fermentative production of biogas, characterized in that a fermentation mixture comprising water, an organic substrate and microorganisms is stirred in a container with a stirrer axially mounted in the container and that fermentation mixture is conveyed via an external conduit into a closed circular pipeline having several spray nozzles and is sprayed across the surface of the fermentation mixture in the container.
